(19) United States
(12) Patent Application Publication
(10) Pub. No.: US 2003/0059956 A1
(43) Pub. Date: Mar. 27, 2003

(54) Method to follow progression of kidney disease

(76) Inventors: Kumar Sharma, Berwyn, PA (US); Tracy A. McGowan, Philadelphia, PA (US); Stephen R. Dunn, Philadelphia, PA (US); Bonita Falkner, Philadelphia, PA (US)

Correspondence Address: THOMAS JEFFERSON UNIVERSITY, INTELLECTUAL PROPERTY DIVISION, 1020 WALNUT STREET, SUITE 620, PHILADELPHIA, PA, 19107, US

(21) Appl. No.: 10/138,962

(22) Filed: May 3, 2002

Related U.S. Application Data
(60) Provisional application No. 60/288,307, filed on May 3, 2001.

(51) Int. Cl. G01N 33/546

(52) U.S. Cl. 436/534

(57) ABSTRACT

The present invention relates to methods of measuring urinary TGF-β levels to monitor disease progression in patients having chronic fibrotic diseases or kidney diseases, more particularly, methods of preparing urine specimens for measuring urinary TGF-β levels in patients having chronic fibrotic diseases or kidney diseases.

### CONTINUING APPLICATION DATA

This application claims priority under 35 U.S.C. §119 based upon U.S. Provisional Patent Application No. 60/288,307 filed on May 3, 2001.

### FIELD OF THE INVENTION

The instant invention is generally related to urology, more particularly, methods of preparing urine specimens for use in measuring TGF-β levels.

### ABBREVIATIONS

ACE means “angiotensin-converting enzyme”.

ARB means “Angiotensin-receptor blocker”.

AT1 means “angiotensin II subtype 1”.

BP means “blood pressure”.

GFR means “glomerular filtration rates”.

TGF-β means “transforming growth factor-β”.

### DEFINITIONS

“Patient,” as used herein, can be one of many different species, including but not limited to, mammalian, bovine, ovine, porcine, equine, rodent and human.

### BRIEF DESCRIPTION OF THE FIGURES

FIG. 1. Correlation between urinary excretion of TGF-β1 and urine protein excretion rates in patients treated with the maximal recommended dose of the ACE inhibitor lisinopril. Horizontal dotted line reflects the upper limit of normal (mean±2 SD) urinary excretion of TGF-β1 in healthy volunteers.

FIG. 2. Correlation between urinary excretion of TGF-β1 and 24-hour systolic ambulatory BP in patients treated with the maximal recommended dose of the ACE inhibitor lisinopril.

FIG. 3. Individual patient data for urinary excretion of TGF-β1 before and after treatment with losartan. There was a 38% reduction (95% Cl, 16 to 55; P=0.017) in urinary TGF-β1 levels from 13.3 (95% Cl, 11.4 to 15.5) to 8.2 (95% Cl, 6.2 to 10.7) pg/mg creatinine.

FIG. 4. Lack of correlation between change in urinary excretion of TGF-β1 and change in urine protein excretion rates in patients treated with add-on losartan therapy ( P=0.84; r²=0.003).

FIG. 5. Lack of correlation between change in urinary excretion of TGF-β1 and change in ambulatory systolic BP in patients treated with add-on losartan therapy ( P=0.575; r²=0.023).

FIG. 6. Urine TGF-β excretion following hyperglycemia.

### DETAILED DESCRIPTION OF THE INVENTION

In the kidney, transforming growth factor-β (TGF-β) appears to be a downstream mediator of several different pathobiologic events and contributes to increased accumulation of extracellular matrix proteins in glomeruli and in the renal interstitium and to progressive renal failure. The physiologic expression of TGF-β, its receptor, and support proteins contributes to the normal function and structure of the nephron, including anti-inflammatory functions and physiologic regulation of renal cell turnover. Thus, TGF-β is probably the single most important molecule in the pathophysiology of renal disease.

Previous studies have shown that urinary TGF-β levels are increased in patients with chronic fibrotic disease and various types of kidney disease, including diabetes, focal sclerosis, and membranous nephropathy. TGF-β levels, therefore, may be used to monitor disease progression in patients having kidney disease or chronic fibrotic disease. In those previous studies, however, urinary TGF-β levels were measured using different methods, resulting in TGF-β levels range from picogram per milligram of creatinine to micrograms per milligram of creatinine. The instant invention provides uniform methods of preparing urine specimens for use in measuring urinary TGF-β levels more accurately and more consistently.

In one embodiment of the instant invention, the urine specimen is prepared by acidifying, neutralizing, and concentrating. More particularly, the urine specimen is prepared by 1) centrifuging an initial volume of urine specimens at room temperature to remove sediments; 2) collecting the supernatants; 3) acidifying the supernatants to a pH of about 2-2.5 with about 1-6 N HCI depending on the volume of the supernatant; 4) neutralizing the supernatants to a pH of about 7.2-7.6 with about 1-6 N NaOH depending on the volume of the supernatant; 5) centrifuging the neutralized supernatants in filter tubes or equivalents, more particularly, Centricon 10 tubes, at about 4° C. for a period of time to remove salts and substrates of molecular weights lower than about 10 kD and to concentrate the specimens to a final volume equal to about 1/10 of the initial volume. The acidified, neutralized, and concentrated urine specimens are then used for determining urinary TGF-β levels using bioassay or immunoassay, such as, but not limited to, ELISA, western blot, and radioimmunoassay. Methods of determining TGF-β levels in vitro using anti-TGF-β antibodies are disclosed in U.S. Pat. No. 5,545,569, incorporated herein by reference. A number of immunoassay kits are available on the market, such as, Quantikine kit for Human TGF-β immunoassay (R&D Systems, Minneapolis, Minn.) and Biotrak ELISA System for Human TGF-β (Amersham Biosciences).

### EXAMPLE 1

### Add-On Angiotensin II Receptor Blockade Lowers Urinary Transforming Growth Factor-· Levels

Progression of renal failure in patients with proteinuric nephropathies, despite renoprotection with angiotensin-converting enzyme (ACE) inhibitors, may be caused by persistent renal production of transforming growth factor-β1 (TGF-β1) through the angiotensin II subtype 1 (AT1) receptors. The hypothesis that AT1- receptor blocker therapy added to a background of chronic maximal ACE inhibitor therapy will result in a reduction in urinary TGF-β1 levels in such patients was tested in this study.

Patients and Methods

Subjects

Patients aged between 18 and 80 years with proteinuria of 1 g/d of protein or greater, hypertension defined as mean arterial pressure of 97 mm Hg or greater, serum potassium level of 5.5 mEq/L or less, and on lisinopril therapy of 40 mg/d for longer than 3 months were eligible for the study. Patients previously administered angiotensin-receptor blockers (ARBs) or with estimated creatinine clearances of less than 30 mumin were excluded. A separate group of 12 healthy volunteers with normal BP, normal albuminuria, and no history of kidney disease or diabetes served as the control group for urinary TGF-β1 levels.

Protocol

The study was a two-period, crossover, randomized, controlled trial. Seventeen (17) patients were administered a sequence of either losartan, 50 mg/d, for 4 weeks, 2-week washout, and placebo for 4 weeks or placebo for 4 weeks, 2-week washout, and losartan, 50 mg/d, for 4 weeks. Lisinopril, 40 mg/d, along with other antihypertensive therapy, was continued throughout the trial. Twenty-four-hour urine was collected for protein, sodium, urea, creatinine, and urinary TGF-β1 measurement. Because standard therapy for patients with proteinuria and renal failure includes ACE inhibitors, ACE inhibitor therapy was not discontinued in any patient enrolled in the study. The maximal dose of lisinopril defined by the manufacturer's recommendations was used.

TGF-β Assay

TGF-β1 in plasma was assayed using a sandwich enzyme-linked immunosorbent assay (Quantikine kit for Human TGF-β1 Immunoassay; R&D Systems, Minneapolis, Minn.). Plasma (platelet poor) was handled exactly as outlined in the Quantikine kit instructions with the acid activation followed by neutralization and finally a 10 fold dilution step.

Urine TGF-β1 measurements were performed as follows: 1) storing urine specimens at −70° C. until use; 2) bringing the urine specimens to room temperature; 3) centrifuging the specimens at room temperature for about 5-10 min at about 2500 rpm to remove sediments; 4) collecting about 2 ml of the supernatants from the specimens; 5) acidifying the supernatants to a pH of about 2-2.5 with about 5 N HCI; 6) neutralizing the supernatants to a pH of about 7.2-7.6 with about 5 N NaOH; 7) centrifuging the neutralized specimens in Centricon 10 tubes at 4° C. for about 1 hour to remove salts and substrates with molecular weight lower than 10 Kd and to concentrate to a volume of equal to or less than 200 μl; 8) diluting the specimens to a volume of 200 μl with R&D Systems' RD51 diluent if needed; 9) measuring TGF-β levels using the Quantikine kit (R&D Systems, Minneapolis, Minn.); and 10) correcting final calculations for concentration.

A Spectramax 250 plate reader (Molecular Devices, Sunnyvale, Calif.) was used to measure the absorbance. Specimens were read at 450 nm with wavelength subtraction of 570 to correct for any physical imperfections in plate. Quantification was achieved using an external duplicate standard of TGF-β1 calibrated along 10 points (0-2000 pg/ml). A standard curve was produced using a 4 parameter logistic fit with SOFTmax® PRO version 1.2 for the Macintosh. Appropriate corrections for concentrations were made and values were expressed as TGF-β1 in picograms per milligram of creatinine. The correlation coefficient for standards was greater than 0.98, and the lowest detectable limit was 0.7 pg/mL in undiluted urine. The intra-assay coefficient of variation was 2.5% ±3.0%, and inter-assay coefficient of variation was 5.6% ±4.2%. Recovery of fortified TGF-β1 in urine was 94%. The linearity (from concentrated samples) are: low end (0.14→1.5 pg/ml), 93% ±10%; mid range (1.6→12.5 pg/mi), 98.4±4.0%; and high (12.6→100 pg/ml), 100±2.5%. The curve fittings for 4 param logit, quadratic, and linear are 0.998, 0.997, and 0.994 respectively. The quality control urine assayed with each batch is 17.40 pg/ml ±5.2 (range 32.7−9.7, n=25). TGF-β expressed as TGF-β in picograms per milligram of creatinine was calculated as follows:

A=TGF-β in neat urine (pg/ml)

B=Creatinine in neat urine (mg/100ml)

$$Urinary TGF \text{-} β ( ng / g creatinine ) = \frac{A pg \times 1 ng \times 100 ml \times 1000 mg}{ml \times 1000 pg \times B mg \times 1 g}$$

Laboratory Analysis

Serum chemistry tests, complete blood counts, urine protein, electrolytes, urea, and creatinine were measured using routine methods. Specifically, creatinine was measured on a Hitachi 911 analyzer (Roche Diagnostic Corp, Indianapolis, Ind.) using the alkaline picrate method, and urine protein was measured by means of a turbidometric method using benzethonium chloride read at 550 nm (Roche Diagnostics Corp). Ambulatory BP monitoring was performed using SpaceLabs 90207 monitors (SpaceLabs, Richmond, Wash.), and GFR measurements were performed with continuous infusion of iothalamate as described in Agarwal R et al., Kidney Int 59: 2282-2289, 2001.

Statistical Analysis

The normality assumption was tested with the Kolmogrov Smirnov statistic, and homogeneity of variances was tested using Lavene's test. Urinary TGF-β and protein excretion were not normally distributed and were log transformed to satisfy the normality assumption. These log-transformed data were used for subsequent analysis. Exponents of the means of logs are reported to reflect geometric means. Data were analyzed by two-way mixed-model analysis of variance with subjects as the random variable and drug or placebo assignment as the fixed variable. Because there were three urine collection periods before losartan exposure, they were averaged and compared with urinary TGF-β excretion after losartan therapy. Bonferroni t-test was used for post hoc testing. All tests were two sided at an a level of 0.05. All statistical analyses were performed using standard procedures on Statistica for Windows, release 5.5 (StatSoft Inc, Tulsa, Okla.).

Results

Baseline Clinical Characteristics

Of the seventeen (17) patients enrolled, sixteen (16) completed the study. One patient could not keep scheduled appointments and was dropped from the study. Causes of renal disease, baseline medications, and other demographic characteristics are listed in Table 1. Nine patients were administered placebo first, and seven patients, the drug first.

Clinical Correlations of Proteinuria and BP With Urinary TGF-β Levels at Baseline

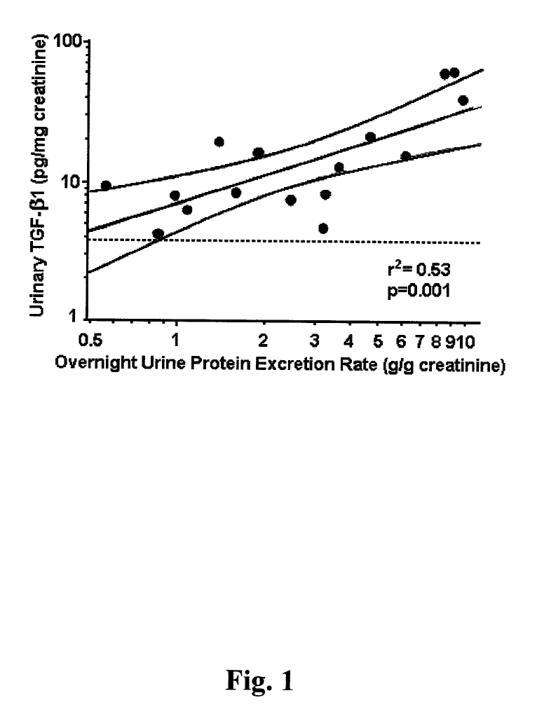

The geometric mean level of urinary TGF-β1 was significantly greater in the study population than the mean value in the group of healthy controls (FIG. 1). Urinary TGF-β1 excretion rate was 13.2±1.2 (SE) pg/mg creatinine in patients, whereas healthy controls had urinary TGF-β1 excretion between 0.2 and 4.1 pg/mg creatinine (P<0.001). Of note, the study population showed increased urinary TGF-β1 levels despite maximal-dose lisinopril therapy.

The mean urinary protein excretion rate in baseline overnight urine specimens was 2.62±1.25 (SE) g/g creatinine. FIG. 1 shows the excellent correlation between baseline proteinuria and urinary TGF-β1 levels (r²=0.53; P=0.001). More than half the variance in baseline urinary TGF-β1 levels was explained by the baseline protein excretion rate.

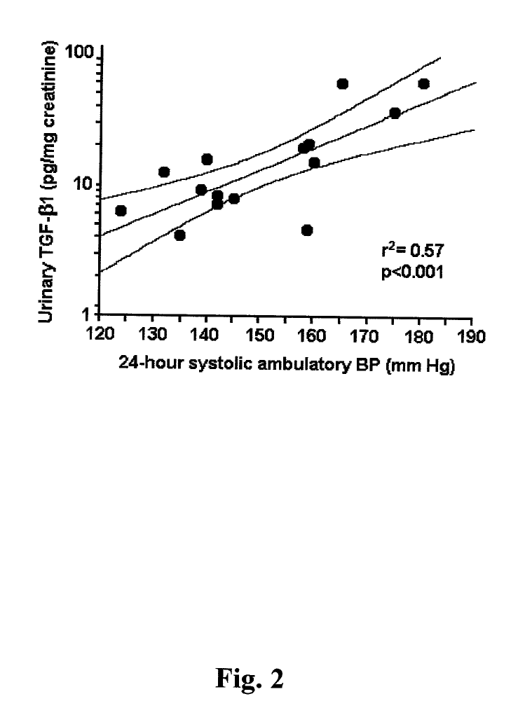

Baseline 24-hour ambulatory systolic BPs also showed a significant correlation with urinary TGF-β1 levels (r²=0.58; P=0.001; FIG. 2). However, baseline diastolic BP (r=0.23; P=0.399), GFR (r=−0.01; P=0.96), and 24-hour urine sodium excretion rate (r=−0.18; P=0.57) did not correlate with urinary TGF-β1 levels.

Effect of Add-On Losartan on TGF-β Levels

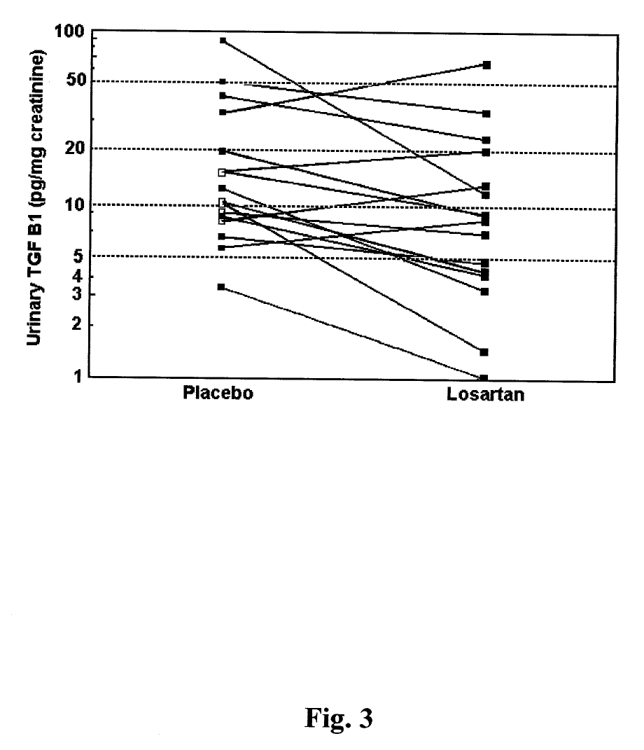

The response to add-on losartan therapy for 4 weeks on urinary TGF-β1 excretion rates is shown in FIG. 3. Average pre-losartan urinary TGF-β1 levels decreased from 13.3 (95% confidence interval [Cl], 11.4 to 15.5) to 8.2 pg/mg creatinine (95% Cl, 6.2 to 10.7) on exposure to losartan, a 38% decrease (95% Cl, 16 to 55; P=0.017).

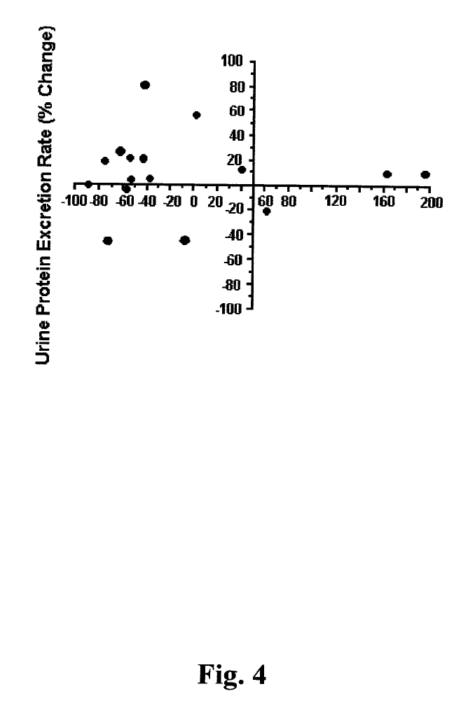

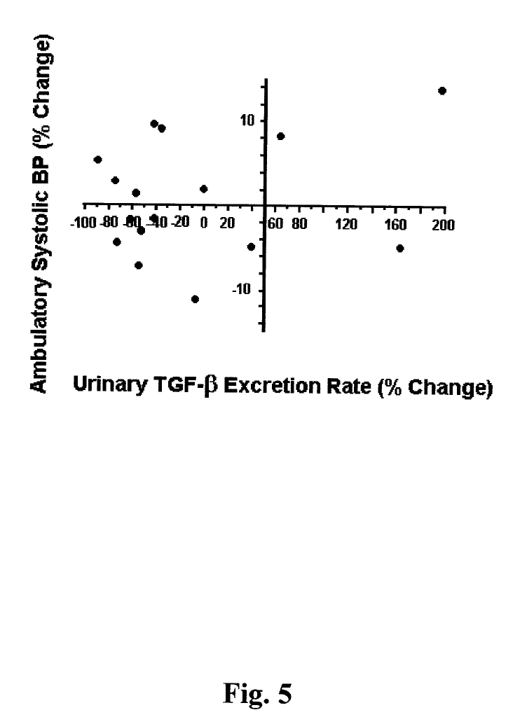

Finally, no relationship between change in proteinuria and reduction in urinary TGF-β1 excretion rate was found (FIG. 4; P=0.84; r²=0.003). Similarly, there was no correlation between change in systolic BP and reduction in urinary TGF-β1 excretion rate (FIG. 5; P=0.575; r²=0.023) or change in diastolic BP and decrease in urinary TGF-β1 excretion rate (P=0.983; r²<0.001).

TABLE 1
Baseline Characteristics

| | |
|---|---|
| Age (y) | 53 ± 9 |
| Men | 14/16 |
| Ethnicity | |
| White | 6 |
| Black | 10 |
| BMI (kg/m 2) | 38 ± 5.7 |
| Cause of CRF | |
| Diabetes | 12 |
| Glomerulonephritides | 4 |
| Antihypertensive medications | |
| Calcium channel blockers | 12 |
| β-Blockers | 6 |
| α-Blockers | 5 |
| Loop diuretics | 10 |
| Thiazide diuretics | 3 |
| No. of antihypertensives | 3.13 ± 1.2 |
| Seated BP (mm Hg) | 156 ± 18/88 ± 12 |
| Seated pulse (beats/min) | 77 ± 11 |
| Standing BP (mm Hg) | 153 ± 22/90 ± 15 |
| Standing pulse (beats/min) | 83 ± 13 |
| Baseline serum creatinine (mg/dL) | 2.0 ± 0.8 |
| Baseline proteinuria (g/g creatinine/24 h) | 3.6 ± 0.71 |

NOTE.
Values expressed as mean 6 SD except valves for proteinuria that are mean ± SEM. All patients were taking lisinopril 40 mg/d.
Abbreviation: CRF, chronic renal failure.

Conlusion

By measuring urinary TGF-β levels using urine specimens prepared as disclosed herein, three major findings were demonstrated by this study: first, patients with chronic proteinuric renal failure have elevated urinary TGF-β1 levels despite maximal lisinopril therapy; second, there is an excellent correlation between TGF-β1 levels in urine and extent of proteinuria and systolic ambulatory BP; and third, there is an early improvement in urinary TGF-β1 levels with losartan therapy.

### EXAMPLE 2

### Hyperglycemia Induces an Increase in Urinary TGF-·1 Excretion in Normal Healthy Volunteers

Cell culture and animal studies, in both rat and mouse models of diabetes, have shown that glucose or hyperglycemia stimulates TGF-β1 production in renal cells. This in vivo study was conducted to determine if increased renal production of TGF-β1 could be detected in normal humans following exposure to 120 min of hyperglycemia.

Methods

A hyperglycemic clamp procedure was performed on fourteen (14) healthy volunteers. An infusion of 20% glucose was delivered at a rate to maintain the plasma glucose between 200-250 mg/dl for 120 min and then tapered to 120 mg/dl over 30 min. Timed urine samples, collected on an overnight period prior to the study, at each void on completion of the procedure, and the following overnight, were assayed for creatinine and TGF-β1. Plasma samples were assayed for TGF-β1 prior to and at 30 min intervals throughout hyperglycemia.

TGF-β1 levels in plasma and urines were measured using Quantikine kit for Human TGF-β1 immunoassay (R&D Systems, Minneapolis, Minn.), as described above.

Results

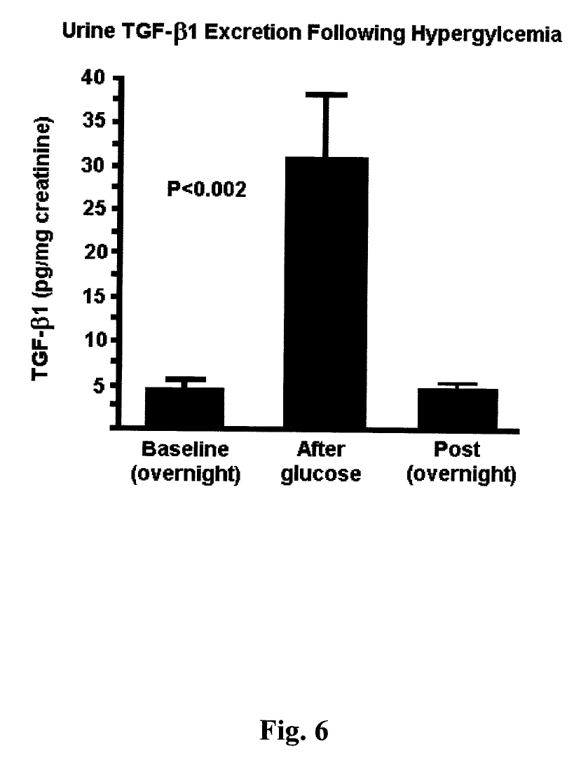

Mean baseline TGF-β1 in plasma was 4.75±0.76 ng/ml. There was no change in plasma TGF-β1 levels throughout the hyperglycemic period. As shown in FIG. 6, baseline urine TGF-β1 (corrected for creatinine excretion) was 4.14±4.03 pg/mg creatinine. The fractional urine samples showed a sharp increase in TGF-β1 excretion in the 12 hr period following exposure to hyperglycemia, with a mean peak TGF-β1 of 30.43±27.88 pg/mg (p=0.0002, non-parametric t-test). A marked spike in TGF-β1 excretion occurred in twelve (12) of fourteen (14) patients. TGF-β1 excretion in the subsequent overnight urine sample was no different from baseline (4.62±4.18 pg/mg).

Conclusion

These results indicate that 120 min of sustained hyperglycemia in normal humans can induce an increase in renal TGF-β1 excretion. In diabetic patients, up-regulation in tissue TGF-β1 activity by hyperglycemia may be an initiating mechanism for the development of diabetic nephropathy.

What is claimed is:

1. A method of preparing urine samples from a patient for use in measuring urinary levels of a TGF-β protein comprising:
a) centrifuging a first volume of urine specimens at room temperature for a first period of time sufficient enough to remove sediments;
b) collecting the supernatants from step a);
c) acidifying the supernatants from step b) with a second volume of HCI enough to bring a first pH to about 2-2.5;
d) neutralizing the supernatants from step c) with a third volume of NaOH enough to bring a second pH to about 7.2-7.5; and
e) centrifuging the neutralized specimens from step d) in an appropriate filter tube for a second period of time sufficient enough to remove salts and substrates of molecular weight lower than 10 kD and to concentrate said neutralized specimens to a final volume equal to about 1/10 of said first volume of urine specimens.

2. The method of claim 1, wherein said HCI has a concentration of about 1-6 N.

3. The method of claim 2, wherein said HCI has a concentration of about 5 N.

4. The method of claim 1, wherein said NaOH has a concentration of about 1-6 N.

5. The method of claim 4, wherein said NaOH has a concentration of about 5 N.

6. The method of claim 1, wherein said appropriate filter tube is a Centricon 10 tube.

7. The method of claim 1, wherein said TGF-β protein is selected from TGF-β1, TGF-β2, or TGF-β3.

8. The method of claim 7, wherein said TGF-β protein is TGF-β1.
